(19) United States
(12) Patent Application Publication
(10) Pub. No.: US 2026/0123820 A1
(43) Pub. Date: May 7, 2026

(54) HANDLE ASSEMBLY FOR USE IN A MEDICAL INSTRUMENT AND MEDICAL INSTRUMENT COMPRISING SUCH A HANDLE ASSEMBLY

(71) Applicant: OLYMPUS Winter & Ibe GmbH, Hamburg (DE)

(72) Inventors: Andreas MUECKNER, Hamburg (DE); Sebastian JUNGBAUER, Hamburg (DE)

(73) Assignee: OLYMPUS Winter & Ibe GmbH, Hamburg (DE)

(21) Appl. No.: 19/381,489

(22) Filed: Nov. 6, 2025

(30) Foreign Application Priority Data
Nov. 7, 2024 (EP) 24 211 324.9

(51) Int. Cl. A61B 1/00

(52) U.S. Cl. CPC A61B 1/00066; A61B 1/00016; A61B 1/00032; A61B 1/0011; A61B 1/00117; A61B 1/00126; A61B 1/00147

(57) ABSTRACT

A handle assembly for use in a medical instrument. The handle assembly including: a sleeve which delimits an internal space thereof and is configured as a structural component; at least one electronic module housed in the internal space of the sleeve; and a heat spreader material configured to spread heat produced in the internal space of the sleeve on an outside of the sleeve. Wherein the heat spreader material is configured as part of an antenna system configured to establish wireless communication between the at least one electronic module and an external communication device.

### CROSS-REFERENCE TO RELATED APPLICATION

The present application is based upon and claims the benefit of priority from EP 24 211 324.9 on Nov. 7, 2024, the entire contents of which is incorporated herein by reference.

### BACKGROUND

### Field

The present disclosure relates to a handle assembly for use in a medical instrument, and more particularly, to a handle assembly for an endoscope, comprising a sleeve which delimits an internal space thereof and serves as a structural component, at least one electronic module housed in the internal space of the sleeve, and a heat spreader material configured to spread heat produced in the internal space of the sleeve on the outside of the sleeve. Furthermore, the present disclosure relates to a medical instrument, and more particularly, to an endoscope, comprising such a handle assembly.

### Prior Art

It is known that inside the sleeves of handle assemblies of medical instruments, heat may be produced which has to be dissipated, for example due to light guides of endoscopes passing through their handle assemblies and in particular fibre couplings being positioned in such handle assemblies which produce a substantial amount of waste heat in small areas such that hot spots inside the handle assembly could occur which might damage its structure and/or impair its functionality.

For these reasons, it is known in the art to position heat spreader materials outside of such sleeves which spread the heat produced in certain small areas or hot spots along the extension of the handle in order to prevent localized high temperatures in the corresponding positions as well as large temperature gradients along the respective handles. Such heat spreader materials have oftentimes been implemented by sheet metals arranged around the sleeve delimiting the internal space of the handle assembly such that any temperature gradient originating from certain points or small areas of heat production inside the sleeve can be distributed and dissipated along the extension of the heat spreader material.

On the other hand, there has been a recent development that certain electronic modules are to be positioned inside the internal space of such handle assemblies, for example sensor(s) with corresponding processing devices which may detect usage states of the respective medical instruments and provide information representing the usage states to an external device. One such use case may for example be accelerometer data gathered by an electronic module located inside the handle assembly which may be monitored in order to detect unwanted drops or impacts on the instrument.

In such configurations and in particular in use cases in which the corresponding at least one electronic module housed in the internal space of the sleeve is also to be operated in configurations in which the medical instrument is not connected to a superordinate or external structure by means of communication lines, it can be necessary to establish a wireless communication between the at least one electronic module and an external communication device by an antenna system.

In cases in which the above discussed heat spreader material made of a metal material is provided in a handle assembly also comprising an antenna system, such an internal antenna would be shielded which would drastically reduce its performance. On the other hand, varying the positioning of such antennas in the handle assembly might result in undefined radiation characteristics thereof.

It also has to be kept in mind in this context that the size of an antenna is primarily determined by its transmission wavelength and the lower the frequency, the larger the dimensions of the antenna have to be. The minimum size of the antenna and its mass surface is determined by the lowest frequency in the application and for this reason, devices with integrated antennas cannot arbitrarily be reduced in size without degradation of antenna performance. It also has to be acknowledged that the size of the required ground plane for such an antenna system should also be around the same size as the actual active part thereof which is in conflict with the available space for using a printed circuit board in handle assemblies of typical medical instruments such as endoscopes.

### SUMMARY

For these reasons, an object is to provide an improved handle assembly which on the one hand allows for spreading heat produced inside its internal space and on the other hand facilitates wireless communication of at least one electronic module housed in its internal space to an external communication device.

Accordingly, a handle assembly for use in a medical instrument, such as an endoscope is provided. The handle assembly comprising a sleeve which delimits an internal space thereof and serves as a structural component, at least one electronic module housed in the internal space of the sleeve, and a heat spreader material configured to spread heat produced in the internal space of the sleeve on the outside of the sleeve, wherein the heat spreader material also serves as part of an antenna system configured to a establish wireless communication between the at least one electronic module and an external communication device. Thus, by integrating the antenna system and the heat spreader material, the reduced space available in such handle assemblies is used optimally, the required heat spreading capacity can be achieved, and wireless communication with the external communication device can become possible in an unobstructed manner.

A main part of the heat spreader material in the handle assembly may serve as a ground plane of the antenna system and a second part thereof may serve as a radiator part of the antenna system. It shall be pointed out that in scenarios in which the corresponding handle assembly is not mechanically and electrically connected to external devices or superordinate structures, in order to operate the antenna system, a ground plane can be provided in the handle assembly with sufficient mass. The main part of the heat spreader material can be made from metal material due to both its terminal conductivity and its capability to serve as a ground plane for the antenna system.

In order to achieve the desired wavelength of the antenna system depending on the dimensions of the antenna system, the heat spreader material may at least partially be formed in a helical shape extending around the sleeve, such that larger antenna lengths are possible. In such embodiments, the heat spreader material may comprise a circular main part serving as the ground plane of the antenna system and a second helical part serving as the radiator part of the antenna system.

In an embodiment, the heat spreader material may comprise a circular part serving as the ground plane of the antenna system and a second part serving as the radiator part of the antenna system may be formed as a coil or a dual inverted F-shaped part.

The heat spreader material itself may be formed as a shaped or slotted metal part and be pushed over the sleeve in order to arrange it at its desired position and/or it may be formed by employing a 3D-MID method.

In order to be able to operate in frequency ranges usually used for wireless communication, the antenna system of the handle assembly may be configured to transmit in a sub-GHz ISM-band at 868 MHz and/or at 2.400 MHz and/or the λ/4-length of the antenna system may be about 83 mm. However, different frequency ranges may be employed as well, depending for example on the type of medical instrument as well as the preferred communication protocol.

As already briefly discussed above, in certain use cases of handle assemblies, the at least one electronic module may be configured for standalone operation, such that it may comprise a battery and/or it may be configured to monitor use conditions of the medical instrument by at least one sensor.

Typically, the sleeve of the handle assembly may at least be partially made from a plastic material which reduces weight and manufacturing costs, a composite material, a ceramic material or a metal material and/or it may further comprise an outer grip portion, which can also be made from a plastic material, which can be positioned outside of the sleeve and the heat spreader material in a radial direction and configured to be gripped by the user of the instrument. Thus, in such handle assemblies, the heat spreader material as well as the antenna system can be positioned between the sleeve and the outer grip portion such that there are no metal structures radially outside of the antenna system potentially blocking antenna radiation and any heat produced inside the sleeve can easily be distributed between the two components in order to prevent any local build-up of heat or hot spots.

Furthermore, also provided is a medical instrument, such as an endoscope, comprising a handle assembly, which may further comprise a light guide extending through the sleeve, for example comprising a fibre coupling assembly positioned at a longitudinal position corresponding to the heat spreader material.

### BRIEF DESCRIPTION OF THE DRAWINGS

Further features and advantages will become even clearer from the following description of embodiments thereof. In the drawings:

FIG. 1 illustrates a schematic cross-section view of a handle assembly; and

FIGS. 2a to 2d illustrate schematic views of different possible configurations of heat spreader materials in the handle assembly of FIG. 1.

FIG. 3 illustrates a medical instrument having the handle assembly of FIG. 1.

### DETAILED DESCRIPTION

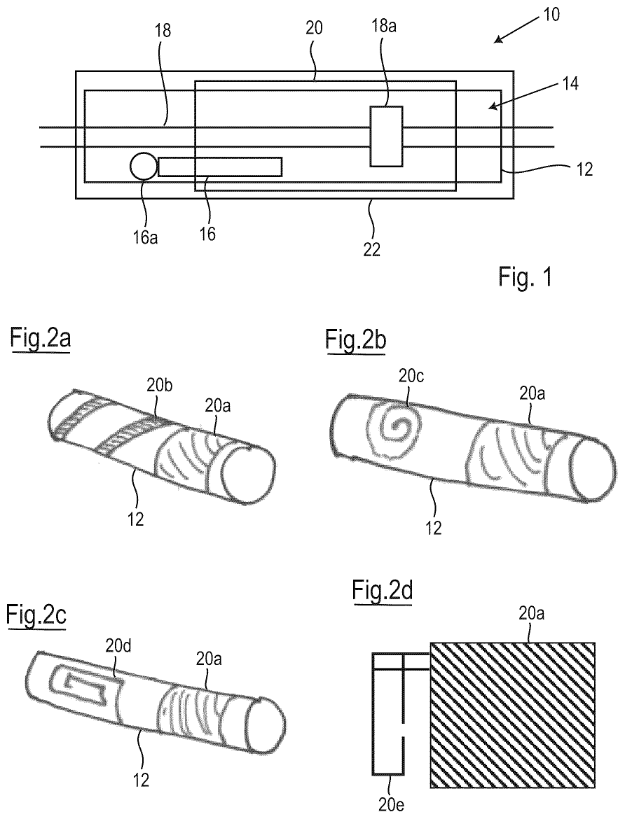

FIG. 1 is a schematic cross-section view showing a handle assembly for use in a medical instrument such as an endoscope which is generally denoted by reference number 10. The handle assembly 10 comprises an inner sleeve 12 which delimits an internal space 14 thereof and serves as a structural component.

Inside the internal space 14 of the sleeve 12 there is provided an electronic module 16, such as a printed circuit board comprising at least one accelerometer as well as a battery 16a for powering the components of the electronic module 16. Furthermore, a light guide 18 extends through the internal space 14 of the sleeve 12, wherein a fibre coupling assembly 18a is provided, by which two parts of the light guide 18 on the right side and on the left side of FIG. 1 can be connected and disconnected for an assembly and disassembly of the handle assembly 10 to a superordinate structure. It is expected that in the region of the fibre coupling assembly 18a, substantial waste heat is produced inside the internal space 14 of the sleeve 12 during operation of the medical instrument which has to be spread or dissipated along the sleeve 12 in order to prevent a local build-up of heat and excessive temperature gradients in the handle assembly 10.

For this purpose, a heat spreader material 20 configured to spread heat produced in the internal space 14 of the sleeve 12 is positioned on the outside of the sleeve 12 in order to spread the heat locally produced inside the sleeve 12 to a wider area. Said heat spreader material 20 for this purpose is made of a metal material which exhibits high thermal conductivity, such as for example copper. Furthermore, said heat spreader material 20 also serves as part of an antenna system configured to establish wireless communication between the electronic module 16 and an external communication device. Radially outside of the heat spreader material 20 and the sleeve 12 there is furthermore provided an outer grip portion 22, which can be made of a plastic material, configured to be gripped by a user of the medical instrument.

Four possible embodiments of such heat spreader material configurations 20 are shown in FIGS. 2a to 2d in schematic views, wherein FIGS. 2a to 2c are isometric views of the respective heat spreader material configurations, while FIG. 2d depicts the corresponding heat spreader material in an unrolled view.

In the embodiment of FIG. 2a, the main part 20a of the heat spreader material 20 positioned at a longitudinal position corresponding to the fibre coupling assembly 18a inside the internal space 14 serves as a ground plane of the antenna system 20 and a second part 20b serving as a radiator part of the antenna system 20 is formed in helical shape extending around the sleeve 12 and in a longitudinal direction thereof.

In alternative embodiments shown in FIGS. 2b and 2c, similar main parts 20a serving as ground planes are provided, however different geometries are used for the respective second parts 20c and 20d, which are in these cases formed as coils with different geometries. Lastly, FIG. 2d shows an embodiment in which the second part 20e serving as the radiator part of the antenna system is formed as a dual inverted F-shaped part which allows for transmitting in different frequency ranges.

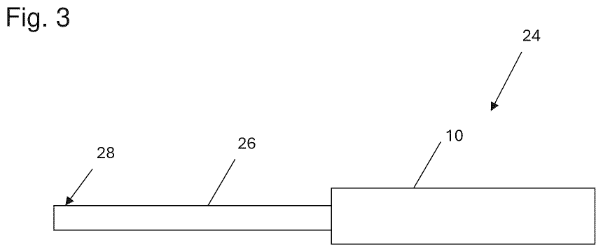

FIG. 3 illustrates a medical instrument 24 having the handle assembly 10 illustrated in FIG. 1 as well as an insertion portion 26, which can be rigid or flexible, and may have an articulating distal end. A distal end 28 of the insertion section 26 may have one or more components comprising an image sensor, illumination means and end-effector. In the medical instrument 24, the light guide 18, video connections as well as mechanical components for operating such components may be installed in one or more of the internal space 14 of the handle assembly 10 and in the insertion portion 26.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A handle assembly for use in a medical instrument, the handle assembly comprising:
a sleeve which delimits an internal space thereof and is configured as a structural component;
at least one electronic module housed in the internal space of the sleeve; and
a heat spreader material configured to spread heat produced in the internal space of the sleeve on an outside of the sleeve;
wherein the heat spreader material is configured as part of an antenna system configured to establish wireless communication between the at least one electronic module and an external communication device.

2. The handle assembly according to claim 1, wherein a main part of the heat spreader material is configured as a ground plane of the antenna system and a second part of the heat spreader material is configured as a radiator part of the antenna system.

3. The handle assembly according to claim 1, wherein the heat spreader material is at least partially formed in a helical shape extending around the sleeve.

4. The handle assembly according to claim 2, wherein the main part is circular and the second part is helical.

5. The handle assembly according to claim 2, wherein the main part is circular and the second part is formed as one of a coil or a dual inverted F-shaped part.

6. The handle assembly according to claim 1, wherein the heat spreader material is formed as one of a shaped or slotted metal part and configured to be pushed over the sleeve.

7. The handle assembly according to claim 1, wherein the heat spreader material is formed by employing a 3D-MID method.

8. The handle assembly according to claim 1, wherein the antenna system is configured to transmit in one of a Sub-GHz ISM band at 868 MHz or at 2400 MHz.

9. The handle assembly according claim 1, wherein the at least one electronic module comprises one of a battery or is configured to monitor use conditions of the medical instrument by at least one sensor.

10. The handle assembly according to claim 1, wherein the sleeve is at least partially made from one of a plastic material, a composite material, a ceramic material or a metal material.

11. The handle assembly according to claim 1, further comprising an outer grip positioned outside each of the sleeve and the heat spreader material in a radial direction and configured to be gripped by a user.

12. The handle assembly according to claim 11, wherein the outer grip is formed of plastic.

13. A medical instrument comprising the handle assembly according claim 1.

14. The medical instrument according to claim 13, further comprising a light guide extending through the sleeve.

15. The medical instrument according to claim 14, wherein the light guide further comprises a fibre coupling assembly positioned at a longitudinal position of the handle assembly corresponding to the heat spreader material.
